(19) United States
(12) Patent Application Publication
(10) Pub. No.: US 2025/0127835 A1
(43) Pub. Date: Apr. 24, 2025

(54) COMPOSITION BASED ON PLANT EXTRACTS AND USE THEREOF FOR THE PREVENTION AND/OR TREATMENT OF CARBOHYDRATE AND LIPID DYSMETABOLISM

(71) Applicant: ESSERRE PHARMA SRL, Roma (RM) (IT)

(72) Inventors: Costanza Valentina RICCIONI, Roma (RM) (IT); Amedeo SQUILLACE GRECO, Roma (RM) (IT)

(73) Assignee: ESSERRE PHARMA SRL, Roma (RM) (IT)

(21) Appl. No.: 18/572,556

(22) PCT Filed: Jun. 27, 2022
(86) PCT No.: PCT/EP2022/067514

§ 371 Date: Dec. 20, 2023

(30) Foreign Application Priority Data
Jun. 25, 2021 (IT) 102021000016787

(51) Int. Cl. A61K 36/33; A61K 9/00; A61K 9/20; A61K 31/51; A61K 31/575; A61K 31/7048; A61K 36/752; A61K 47/02; A61K 47/12; A61K 47/36; A61P 3/06

(52) U.S. Cl. CPC A61K 36/33; A61K 9/0095; A61K 9/2009; A61K 9/2013; A61K 9/2054; A61K 31/51; A61K 31/575; A61K 31/7048; A61K 36/752; A61K 47/02; A61K 47/12; A61K 47/36; A61P 3/06

(57) ABSTRACT

The present invention relates to a composition containing Opuntia ficus indica extract and at least one substance selected from among Bergamot extract (Citrus Bergamia) and/or Lemon and/or Orange and/or Pomelo and/or Grapefruit, preferably Bergamot extract, phytosterols, and at least one trace element.

### FIELD OF THE INVENTION

The present invention relates to a composition based on multiple plant extracts and the use thereof in the pharmaceutical field or as a food supplement in the prevention and treatment of risk factors related to carbohydrate and lipid dysmetabolism.

### BACKGROUND ART

Cardiovascular diseases (diabetes mellitus, stroke, heart attack, atherosclerosis, etc.) are the leading cause of morbidity throughout the world, particularly in Western and more “developed” nations, but with an increasing number of cases in developing countries as well. The correlation between plasma LDL cholesterol levels and cardiovascular risk has been amply demonstrated in both observational and interventional studies.

The pharmacological therapy of choice for dyslipidemia is represented by statins, inhibitors of the enzyme hydroxymethylglutaryl-coenzyme A-reductase (HMG-CoA-reductase). These act by inhibiting the endogenous biosynthesis of cholesterol in the liver, blocking the conversion of 3-hydroxy-3-methylglutaryl-CoA (HMG-CoA) to mevalonic acid, the precursor of cholesterol.

Commercially available molecules are simvastatin, lovastatin, pravastatin, atorvastatin, fluvastatin, and rosuvastatin.

Statins can be considered safe medicines; the main side effects are alterations in liver function, effects on muscles (myalgia and myopathy) and, more rarely, rhabdomyolysis.

The most common manifestations of side effects such as myalgia and myopathy and the condition of statin intolerance are considered the main causes of therapy discontinuation.

Other drugs used in the treatment of dyslipidemia include fibrates, ion exchange resins, ezetimibe. Type II diabetes mellitus is a clinical condition characterized by a combination of impaired insulin secretion and peripheral insulin resistance. The consequence of a lack of compensation with increased insulin production by the beta cells of the pancreas is an increase in blood glucose that can lead to the development of complications such as retinopathy, nephropathy, neuropathy and atherosclerosis. Around 415 million people worldwide suffer from diabetes mellitus, a number which is expected to increase to 650 million by 2040.

The drug of choice in the treatment of diabetes is metformin (biguanide class), which improves the sensitivity of tissues to insulin. The mechanism of action of metformin seems to comprise the activation of AMPK (activated protein kinase adenosine monophosphate) which plays a fundamental role in the body's energy balance by regulating carbohydrate and lipid metabolism.

Side effects of metformin are quite common and include nausea, vomiting, diarrhoea, constipation, flatulence and, more rarely, lactic acidosis.

Other oral antidiabetics used in the treatment of type II diabetes include sulfonylureas, glinides, glitazones, DPP-4 inhibitors, intestinal alpha-glucosidase inhibitors, SGLT-2 renal glucose transporter inhibitors.

All of these drugs have almost the same side effects as metformin, some of which may also cause weight gain.

NAFLD (“Non-alcoholic Fatty Liver disease”) is the most common liver disease in Western countries, with a sharp increase in recent years in developing countries as well. It is a condition closely related to obesity and insulin resistance and frequently present in patients with diabetes who are more likely to develop NASH (“Non-Alcoholic-Steato Hepatitis”) and cirrhosis of the liver. The most important therapeutic strategy for this disease is weight loss obtained through diet and physical activity. The drugs used include antidiabetic drugs (pioglitazone) and antiobesity drugs, including endocannabinoid receptor antagonists, but characterized by CNS side effects (depression and psychiatric disorders).

Considering the multifactorial etiology of cardiovascular diseases, many patients require polytherapy, often consisting of antihypertensive, antidiabetic, and lipid-lowering drugs. This can more often lead to the onset of side effects and the abandonment of therapies.

The role of lifestyle (proper nutrition and physical activity) in cardiovascular prevention and in the fight against risk factors is known.

The use of so-called “nutraceuticals”, foods or substances of plant or natural origin which are concentrated sources of nutrients or promote the physiological functions of the body is a useful tool, as part of a proper lifestyle.

Nutraceuticals can play a role in reducing disease risk factors, helping to improve the therapeutic response of patients, also potentially delaying or cancelling the need for the use of pharmacological therapies.

Nutraceuticals, or food supplements, are framed at the regulatory level as food products intended to supplement the common diet and which constitute a concentrated source of substances having a nutritional or physiological effect, and presented in pre-dosed oral, solid or liquid forms such as capsules, tablets, sticks, sachets (Directive 2002/46 EC).

Food supplements are therefore, as foods, generally considered safe, but the occurrence of adverse reactions from natural substances is indeed known, in particular products of plant origin (“botanicals”) but also vitamins or minerals if taken in excess (epicentro.iss.it/fitosorveglianza).

A naturally-occurring ingredient known for its cholesterol-lowering effect is fermented red rice extract (Cicero A F G, Fogacci F, Banach M. Red Yeast Rice for Hypercholesterolemia. Methodist Debakey Cardiovasc J. 2019 July-September; 15(3):192-199. doi: 10.14797/mdcj-15-3-192. PMID: 31687098; PMCID: PMC6822657).

It is a product obtained from the fermentation of red rice by a yeast, Monascus purpureus, and some substances are produced from the fermentation process including monacolin k, which at the molecular level is structurally identical to lovastatin, a synthetic statin, but functionally more similar to the bond with the enzyme HMG-CoA reductase and therefore potentially more effective than the drug itself. This product has been used for years as an ingredient in food supplements sold worldwide but recently, following the publication of some scientific papers that questioned its safety, in particular for the onset of liver and musculoskeletal side effects (Mazzanti G, Moro P A, Raschi E, Da Cas R, Menniti-Ippolito F. Adverse reactions to dietary supplements containing red yeast rice: assessment of cases from the Italian surveillance system. Br J Clin Pharmacol. 2017 April; 83(4):894-908. doi: 10.1111/bcp.13171. Epub 2017 Jan. 19. PMID: 28093797; PMCID: PMC5346868), the European Food Safety Authority (EFSA) published a Scientific Opinion in 2018 in which it states that it is not able to identify a dosage of monacolin k which is completely safe due to the absence of side effects.

Therefore, it is clear that it is necessary to identify new compositions of natural origin, characterized by a high safety of use, which have a wide spectrum of action and are effective in the reduction of cardio-metabolic risk factors, for all those individuals who cannot or are not willing to follow a pharmacological therapy, as in the case of subjects in primary prevention or those intolerant to the pharmacological therapies of choice, the elderly or patients in pharmacological polytherapy, for whom a higher level of alertness is necessary for possible pharmacokinetic and pharmacodynamic interactions.

### SUMMARY OF THE INVENTION

The present invention relates to a composition of naturally occurring extracts characterized by a favourable profile of safety and efficacy in reducing risk factors related to the development of chronic cardio-metabolic disease.

The present invention relates to a composition comprising an extract of Opuntia ficus indica and at least one of the following elements:
An extract of Bergamot or Lemon or Orange or Pomelo or Grapefruit or mix,
A source of plant sterols or stanols and
A trace element (such as Vitamin or Mineral).

The composition object of the invention, preferably formulated in solid, semi-solid or liquid form, is also applicable individually or concomitantly with a specific drug therapy, in the treatment of cardio-metabolic diseases, dyslipidaemias, insulin resistance, type II diabetes, NAFLD (non-alcoholic fatty liver disease)/NASH (non-alcoholic steato hepatitis) and/or hypertension.

An object of the invention is a composition comprising Opuntia ficus indica extract and at least one substance selected from the group consisting of:
Bergamot (Citrus Bergamia) and/or Lemon and/or Orange and/or Pomelo and/or Grapefruit extract, preferably Bergamot extract,
plant sterols or stanols, preferably phytosterols,
and at least one trace element.

Therefore an object of the present invention is a composition comprising Opuntia ficus indica extract and at least one substance selected from the group consisting of:
Bergamot (Citrus Bergamia) and/or Lemon and/or Orange and/or Pomelo and/or Grapefruit extract, preferably Bergamot extract,
phytosterols,
and at least one trace element.

Preferably the composition comprises Opuntia ficus indica extract and Citrus Bergamia extract or Opuntia ficus indica extract and phytosterols.

Preferably the composition comprises Opuntia ficus indica extract, Citrus Bergamia extract and phytosterols.

Preferably the composition comprises Opuntia ficus indica extract, Citrus Bergamia extract, phytosterols and at least one trace element.

Preferably the composition comprises Opuntia ficus indica extract, Citrus Bergamia extract, phytosterols and trace elements.

Preferably the Citrus Bergamia extract comprises brutieridin and melitidin.

Preferably the Opuntia Ficus Indica extract derives from cladodes, flowers, fruits or combinations thereof, preferably it derives from cladodes.

Preferably the Opuntia ficus indica extract comprises:
at least 20% weight/weight of fiber, preferably of which at least 18% of soluble fiber, or
at least 70% of soluble fiber, preferably at least 75%, preferably wherein said soluble fiber comprises 3-5% of beta-polysaccharides and 3-5% of polyphenols.

Preferably the phytosterols comprise about 80% of beta-sitosterol and beta-sitostanol, 6-10% of campesterol, 0.5-1% of stigmasterol, 0.5-1% of delta-7-stigmasterol, 2% of campestanol. Preferably the trace element comprises or is at least one vitamin and/or at least one mineral, preferably the trace element comprises or is thiamine and/or its salts, such as thiamine hydrochloride.

Preferably the composition comprises Opuntia ficus indica extract from 5 to 20% w/w.

Preferably the composition comprises Citrus Bergamia extract from 8 to 20% w/w.

Preferably the composition comprises phytosterols from 10 to 40% w/w.

Preferably the composition comprises thiamine hydrochloride from 0.5 to 1% w/w.

Preferably the composition comprises
a. Opuntia ficus indica extract from 5 to 20% w/w
b. Citrus Bergamia extract from 8 to 20% w/w
c. Phytosterols from 10 to 40% w/w and
d. Thiamine hydrochloride from 0.5 to 1% w/w.

Preferably the composition is in solid, semi-solid or liquid form and/or is preferably in combination with one or more excipients selected from the group comprising: calcium phosphate, magnesium stearate, silicon dioxide, hydroxypropylcellulose, mono and diglycerides of fatty acids, microcrystalline cellulose, glazing agents, maltodextrins, acidifiers of which preferably citric acid, preservatives of which preferably sodium benzoate and/or potassium sorbate, sweeteners of which preferably steviol glycosides, dyes of which preferably iron oxide; and/or in combination with other ingredients suitable for food or pharmaceutical use, such as one or more vitamins, minerals, enzymes, proteins, and/or other plant extracts.

In the composition of the invention preferably the Opuntia ficus indica extract is present in an amount from 150 mg to 1000 mg, preferably 150 mg, 300 mg or 500 mg or 1000 mg.

In the composition of the invention preferably the Citrus Bergamia extract is present in an amount from 200 mg to 500 mg, preferably 200 mg, 400 mg or 500 mg.

In the composition of the invention preferably the phytosterols are present in an amount from 400 to 2000 mg, preferably 400 mg, 800 mg or 2000 mg.

In the composition of the invention preferably thiamine hydrochloride is present in an amount from 10 to 25 mg, preferably 12.5 mg or 25 mg.

In the composition of the invention preferably:
a. Opuntia ficus indica extract is present in an amount from 150 mg to 1000 mg, preferably 150 mg, 300 mg or 1000 mg;
b. Citrus Bergamia extract is present in an amount from 200 mg to 500 mg, preferably 200 mg, 400 mg or 500 mg
c. Phytosterols are present in an amount from 400 to 2000 mg, preferably 400 mg, 800 mg or 2000 mg;
d. Thiamine hydrochloride is present in an amount from 10 to 25 mg, preferably 12.5 mg or 25 mg.

Another object of the invention is a pharmaceutical composition comprising the composition as defined herein and at least one pharmaceutically acceptable excipient and/or vehicle.

A further object of the invention is a dietary supplement or food product or drinking product comprising the composition as defined herein.

Another object of the invention is the composition or the pharmaceutical composition or the dietary supplement or food product or drinking product as defined herein for medical use, preferably for use in the reduction and/or prevention of risk factors related to the development of cardio-metabolic diseases, preferably for use in the reduction of total and LDL cholesterol, triglycerides and/or blood glucose and/or to increase HDL cholesterol, preferably for use in the prevention and/or treatment of dyslipidemia, hypercholesterolemia, hypertriglyceridemia, diabetes associated with dyslipidemia, metabolic syndrome, statin-induced myalgia or myopathy, intolerance to lipid-lowering drugs, clinical conditions characterized by low HDL cholesterol levels, atherosclerosis, insulin resistance, type II diabetes, NAFLD (non-alcoholic fatty liver disease)/NASH (steatohepatitis non-alcoholic), hypertension, obesity, visceral fat and/or for counteracting disorders/risk factors typical of obesity.

Another object of the invention is a non-therapeutic use of the composition or of the dietary supplement or food product or drinking product as defined herein in the nutraceutical sector or as a basic ingredient in preparations of supplements or drugs and/or as an agent for the prevention and/or treatment of carbohydrate and/or lipid dysmetabolism.

### DETAILED DESCRIPTION OF THE INVENTION

Opuntia ficus indica

Opuntia ficus indica (L.) Miller (here also called Opuntia or Opuntia ficus indica) is a dicotyledonous angiosperm plant belonging to the Cactaceae family, which grows in the tropical and subtropical areas of the planet. The major crops of Opuntia are in Mexico (70%), the southern United States, Spain and Italy (3.3%) (Diaz et al., “Opuntia spp.: Characterization and Benefits in Chronic Diseases.” Oxidative medicine and cellular longevity vol. 2017 (2017): 8634249. doi:10.1155/2017/8634249).

The bioactive components of Opuntia are polyphenols (phenols, flavonoids, betaxanthins, betacyanines), vitamins C and E, polyunsaturated fatty acids, amino acids.

The health effects of Opuntia are attributed to both the seeds and fruit, but especially to the cladodes.

The health properties attributed to Opuntia are:
Antiulcer/gastroprotective
Antidiabetics/hypoglycaemic agents
Antiatherogenic/antioxidant/lipid lowering

The hypoglycaemic properties seem to be related to the fibrous component (soluble fibers) and therefore to pectins and mucilages which exert a mechanical effect of binding the sugars consumed with the diet and therefore reducing their absorption and increasing faecal excretion. As for the hypocholesterolemic properties, they seem attributable not to a mechanical but metabolic action, of interference with the pathways linked to the metabolism of cholesterol and therefore partly due to the pectin component and partly to the polyphenolic one, also considered responsible for the anti-atherogenic activity (Diaz et al., 2017; El-Mostafa et al., Nopal cactus (Opuntia ficus-indica) as a source of bioactive compounds for nutrition, health and disease. Molecules. 2014 Sep. 17; 19(9):14879-901. doi: 10.3390/molecules190914879. PMID: 25232708; PMCID: PMC6270776).

Opuntia is also rich in minerals, particularly calcium and potassium.

Some preclinical and clinical studies have described the lipid-lowering and glucose-lowering properties of Opuntia derivatives.

In a clinical study (Giglio et al., 2020 Metabolites 10(428):428; DOI:10.3390/metabo10110428) 500 g of pasta weekly with the addition of 3% Opuntia titrated at 30% in insoluble polysaccharides, after 4 weeks reduces small-dense LDL in subjects with risk factors for metabolic syndrome.

In the study of Godard et al., 2010 (Godard M P, et al. Acute blood glucose lowering effects and long-term safety of OpunDia supplementation in pre-diabetic males and females. J Ethnopharmacol. 2010 Aug. 9; 130(3):631-4. doi: 10.1016/j.jep.2010.05.047. Epub 2010 Jun. 4. PMID: 20621660), taking 200 mg of an Opuntia extract (OpunDia®) significantly reduces blood glucose in the glucose tolerance test.

Nunez-Lopez et al. (Nunez-Lopez M A, Paredes-López O, Reynoso-Camacho R. Functional and hypoglycemic properties of nopal cladodes (O. ficus-indica) at different maturity stages using in vitro and in vivo tests. J Agric Food Chem. 2013 Nov. 20; 61(46):10981-6. doi: 10.1021/jf403834x. Epub 2013 Nov. 12. PMID: 24164385), describes the postprandial glucose lowering effects of an Opuntia cladode extract (50 mg/kg) on animal model (streptozocin-induced diabetic rats).

Phil-sun-Oh et al., 2006 (Biological & Pharmaceutical Bulletin 29(7):1391-6; DOI:10.1248/bpb.29.1391), describes the effects on Triton WR-1339 mice of a 90 kDa glycoprotein from Opuntia in reducing cholesterol (TC and LDL-c) and triglycerides.

### Bergamot

To date, the interest in bergamot (here also called Citrus Bergamia Risso et Poiteau or Citrus Bergamia or C. Bergamia) concerns, in addition to the extraction of the essence from the epicarp, the activity of the polyphenols extracted from the fruit on the maintenance of the physiological values of the cholesterol, triglycerides and glycemia plasma levels. Flavonoids have been discovered in the juice of Citrus bergamia Risso et Poiteau, exclusive of this species of the genus Citrus, called Brutieridin and Melitidin (Di Donna L et al, J Nat Prod, 2009, 72, 1352-1354).

Such compounds are derived from the flavonoids Hesperetin and Naringenin (aglycones). In particular, they are derived from the glycosylated forms (neohesperidosides) of Hesperetin and Naringenin which are called, respectively, Neohesperidin and Naringin.

Brutieridin and Melitidin have a 3-hydroxy-3-methylglutaryl (HMG) moiety linked to the glycoside moiety of the flavonoid.

Brutieridin is the name of the compound 3-hydroxy-3-methylglutaryl derived from Neohesperidin; Melitidin is the name of the compound 3-hydroxy-3-methylglutaryl derived from Naringin.

The presence in these flavonoids of the 3-hydroxy-3-methylglutaryl moiety is responsible for the hypocholesterolemic action, through the inhibition on the natural substrate (HMG CoA) of the enzyme Hydroxymethylglutaryl coenzyme A reductase (HMG-CoA reductase) (Leopoldini M et al., J, Agric. Food Chem. 2010, 58, 10768-10773).

International patent application WO2010/055490 relates to a process for producing a phytocomplex consisting of the dry extract from the albedo of the bergamot fruit and to its use in the pharmaceutical sector as an anti-dyslipidemic and/or anti-atherogen and as a food supplement.

In particular, the phytocomplex obtained by the described method is characterized by the following percentages of bioflavonoids:
Neoeriocitrin 29.6%+/−6.0,
Naringin 32.4%+/−4.0,
Neohesperidin 38.0%+/−6.0
and has a minimum content of the flavonoids listed of not less than 25%.

Miceli N., Journal of Agricultural and food Chemistry, 2007, 55, 10671-10677 describes that the administration of bergamot juice to hyperlipidemic mice results in a reduction in serum cholesterol, triglycerides and LDL, together with an increase in HDL.

A further study (Mollace V et al., Phytotherapy, 2011 April; 82(3):309-16) describes the administration of bergamot juice in both animal and human models. The human study was conducted on 237 patients, divided into groups between patients with isolated hypercholesterolaemia, mixed hyperlipidemia (high cholesterol and triglycerides) and metabolic syndrome (patients with high cholesterol, triglycerides associated with hyperglycaemia) who were treated daily for 30 days with bergamot juice or placebo. The treatment with the polyphenolic fraction derived from bergamot juice led to a reduction in total and LDL cholesterol, triglycerides, blood glucose (depending on the groups) and an increase in HDL. The placebo-treated groups did not achieve appreciable results. The polyphenolic fraction of bergamot used contained 26-28% of the 5 main flavonoids. The content of the 5 main flavonoids was:
neoeriocitrin (7.7%±0.4%), naringin (6.3%±0.33%), neohesperidin (7.2%±0.35%), melitidin (1.56%±0.11%) and brutieridin (3.32%±0.17%).

### Plant Sterols and Stanols

Phytosterols (commonly referred to as plant sterols and stanols) are lipophilic compounds in the family of triterpenes, bioactive components of plant cell membranes. They are characterized by a chemical structure similar to that of cholesterol, consisting of a tetracyclic ring and a long flexible side chain at the C-17 carbon atom, but differ therefrom in the identity of the C-17 side chain.

Plant stanols are characterized by the absence of the double bond Δ-5 on the sterol ring. Plant sterols and stanols are not synthesized endogenously in humans, but are ingested through the diet. Natural sources of phytosterols are for example vegetable oils, grains, nuts, vegetables, fruit, soy. Different mechanisms have been identified which, acting on different phases, can explain the cholesterol-lowering effect of phytosterols.

Firstly, due to their greater lipophilicity, phytosterols have greater affinity of cholesterol for the incorporation in mixed micelles.

Reducing the concentration of micellar cholesterol results in a lower uptake of cholesterol by the enterocyte.

Secondly, phytosterols interfere with the normal mechanisms responsible for the maintenance of intracellular cholesterol homoeostasis, which depends on various factors including the activity of the ABC transporters.

In fact, recent studies have shown that phytosterols are able to activate Liver X nuclear receptors in enterocytes, increasing the cholesterol efflux mediated by the ABC transporters in the intestinal lumen. Since the two transporters ABCG5/ABCG8 are also present in the liver, it is conceivable that the beneficial effect of the phytosterols on cholesterol is also due to an increase in its excretion in bile.

Numerous clinical studies have shown a decrease in LDL-cholesterol levels of around 8-9%, related to the intake of products with added phytosterols (2 g/day) (Helena Gylling, Jogchum Plat, Stephen Turley, Henry N. Ginsberg, Lars Ellegard, Wendy Jessup, Peter J. Jones, Dieter Lutjohann, Winfried Maerz, Luis Masana, Gunther Silbernagel, Bart Staels, Jan Borén, Alberico L. Catapano, Guy De Backer, John Deanfield, Olivier S. Descamps, Petri T. Kovanen, Gabriele Riccardi, Lale Tokgözoglu, M. John Chapman, 2014, “Plant sterols and plant stanols in the management of dyslipidaemia and prevention of cardiovascular disease”. The European Atherosclerosis Society Consensus Panel on Phytosterols. Atherosclerosis 232, 346-360). However, there are conflicting studies, according to which in healthy subjects, a low or high intake of plant sterols did not affect plasma LDL cholesterol concentrations despite the modulation of cholesterol metabolism (Lin X, et al. Eur, J Clin Nutr 2010; 64; 1481-7). Other studies suggest that triglyceride levels are reduced by 6-4% with an intake of 1.6-2.5 g/day of plant stanols (Demonty I., et al. Eur, Jç Nutr 2013; 52; 153-60; Naumann E. et al. J, Am Coll Nutr 2008, 27: 117-26).

Data obtained from clinical trials evaluating the association of phytosterol-added products with statin drug therapy are relevant.

The results show, in the combination of phytosterols and statins, a further decrease in LDL-cholesterol levels of 10-15%, higher than that obtained by doubling the dose of statin (6%) (Gylling et al., 2014); this evidence suggests an additive effect of phytosterols to that exerted by statins, and this by virtue of the difference in the mechanisms of action with which these molecules act, one (phytosterols) inhibiting intestinal absorption, the other (statins) intervening on cholesterol biosynthesis.

### Thiamine

Thiamine (Vitamin B1) was discovered in 1926 by two German scientists and first synthesized by Dr Williams. It is a water-soluble vitamin which plays an important role in numerous biochemical processes, mainly by playing the role of cofactor in the metabolism of carbohydrates and amino acids (Polegato et al., 2019 Role of Thiamine in Health and Disease, Micronutrients, Volume 34, Issue 4, August 2019, Pages 558-564).

There are two sources of Thiamine: dietary intake and bacterial production, the latter being very modest and not comparable to dietary intake.

Food sources of thiamine are rice and (unprocessed) wheat, milk, eggs, beef and pork, fish, nuts, seeds, orange juice, tomato juice, leafy green vegetables.

Thiamine is absorbed in the jejunum and ileum with passive and active absorption.

One study (Waheed P, Naveed A K, Ahmed T. Thiamine deficiency and its correlation with dyslipidaemia in diabetics with microalbuminuria. J Pak Med Assoc. 2013 March; 63(3):340-5. PMID: 23914634.) demonstrates that subjects with diabetes have reduced levels of Thiamine and that Thiamine levels are inversely correlated with those of LDL cholesterol while positively correlated with HDL-c levels.

Thiamine deficiency is also related to several cardiovascular diseases including Type I and II diabetes, obesity, dyslipidemia, myocardial infarction, arrhythmias (Eshak et al., Thiamine deficiency and cardiovascular disorders. Nutr Metab Cardiovasc Dis. 2018 October; 28(10):965-972. doi: 10.1016/j.numecd.2018.06.013. Epub 2018 Jun. 22. PMID: 30143411).

The treatment of Thiamine deficiency is simple, safe, inexpensive and life-saving (Polegato et al.). In a study (Sarandol et al., 2020, Effects of thiamine treatment on oxidative stress in experimental diabetes. Bratisl Lek Listy. 2020; 121(3):235-241. doi: 10.4149/BLL_2020_036. PMID: 32115983) on animal model of streptozocin-induced diabetic rats, the administration of 6 mg/kg of Thiamine for 5 weeks reduces serum glucose, total cholesterol, triglycerides, malonyldialdehyde and increases insulin and HDL-c.

In a clinical trial (Al-Attas O, Al-Daghri N, Alokail M, Abd-Alrahman S, Vinodson B, Sabico S. Metabolic Benefits of Six-month Thiamine Supplementation in Patients With and Without Diabetes Mellitus Type 2. Clin Med Insights Endocrinol Diabetes. 2014 Jan. 23; 7:1-6. doi: 10.4137/CMED.S13573. PMID: 24550684; PMCID: PMC3921172.) in 86 subjects of which 60 with type II diabetes mellitus, the administration of 100 mg of Thiamine per day for 6 months significantly reduces total cholesterol after 3 months of supplementation.

A 300 mg daily dose of Thiamine reduces diastolic and systolic pressure in hyperglycaemic subjects (Alaei-Shahmini et al., 2015, Diabetes and Metabolic Syndrome Clinical Research and Reviews 9(4); DOI:10.1016/j.dsx.2015.04.014).

A maximum daily dose of Thiamine of 25 mg is allowed in food supplements, equal to 1 quarter of the minimum dose considered effective in reducing cardio-metabolic risk factors.

The present composition is capable of inhibiting HMG-CoA reductase and/or inhibiting the intestinal absorption of cholesterol and/or exerting a mechanical binding effect of sugars consumed with the diet and/or facilitating carbohydrate and lipid metabolism. Such effects are greater with respect to those obtained with the individual extracts of the composition. A synergistic effect is thus obtained with the present composition.

In the context of the present invention, the percentages w/w (% w/w) are to be understood as percentages by weight on (or with respect to) the total weight of the composition (or compound), unless contrary or different stated.

Preferably, said composition or pharmaceutical composition or food supplement is administered orally, preferably once or twice per day.

In the context of the present invention, the terms “preparation” or “composition” are used interchangeably.

In the context of the present invention, the terms “supplement” or “dietary supplement” or “food supplement” are used interchangeably.

The Bergamot (Citrus Bergamia) and/or Lemon and/or Orange and/or Pomelo and/or Grapefruit and/or Chinotto extract, preferably Bergamot extract can be prepared with any method known to the person skilled in the art, for example by mixing plant extracts (e.g., bergamot) obtained from different matrices: juice and/or pastazzo (residues of skins, pulp and seeds after pressing the fruit and eliminating the epicarp) and/or albedo with different extraction methods known to the person skilled in the art (e.g., resins and membranes, KOH, EtOH/H₂O/supercritical CO₂); or mixing different citrus extracts (e.g., bergamot and/or grapefruit and/or chinotto and/or bitter orange and/or pomelo or combinations thereof). Preferably the plant extract is or derives from juice, for example obtained by pressing ripe fruit, after eliminating the epicarp. The juice obtained by squeezing can be subjected to an enzymatic process for the elimination of the pectins. The liquid thus obtained is preferably subjected to a first membrane concentration process. The eluate is then preferably subjected to passage in adsorbent resins. The retentate of the resin is preferably washed with a mixture of water and ethanol and the liquid obtained is preferably subjected to solvent removal and a new membrane or heat concentration process. Lastly, the product is preferably dried by spray drying.

The Opuntia ficus indica extract can be prepared by any method known to the person skilled in the art. Preferably, the extract is obtained from the cut cladodes of young plants in good vegetative state. The compound is preferably processed with the addition of water and enzymes and preferably subjected to depulpation and preferably to a subsequent ultrafiltration step. The filtration permeate is preferably passed in adsorbent resins.

In the context of the present invention, the titration of the extracts and the composition is preferably by means of HPLC (high performance liquid chromatography).

Alternatively, after a washing step, the cladodes are subjected to a milling process and then extracted with water (preferably 1:7 ratio cladodes-water), preferably at a temperature comprised between 15 and 18° C., preferably for 12-20 hours, for example 16 hours. A filtration, centrifugation and vacuum concentration step is then carried out. The obtained product is then subjected to precipitation (aqueous extract/isopropanol ratio is preferably 1:3), centrifugation, washing, vacuum drying preferably at a temperature of 70° C. The obtained powder preferably has variable dimensions around 75-100 microns, and preferably consists of (or comprises or is composed of) at least 75% weight/weight soluble fiber (mucilage and pectin), and preferably contains about 3.7% beta-polysaccharides and 3.7% polyphenols.

Preferably the Opuntia ficus indica extract contains an amount of polyphenols greater than 1% w/w and less than 30% w/w.

The Opuntia Ficus Indica extract can be derived from cladodes, flowers, fruits or combinations thereof. Preferably it derives from cladodes.

The Opuntia Ficus Indica extract and the bergamot extract are present in the composition according to the invention in a weight ratio between: 5:1 and 1:5.

The bergamot extract (here also called Citrus Bergamia Risso et Poiteau extract) according to the invention is preferably a dry, or dried, Citrus Bergamia Risso et Poiteau juice extract.

In Citrus Bergamia Risso et Poiteau extract, the flavonoids brutieridin and melitidin preferably form at least about 12.5% of the total flavonoid fraction of the extract.

The process of obtaining the dry or dried Citrus Bergamia Risso et Poiteau juice extract must be such that such an extract has the features described above.

The phytosterols in the composition preferably come in the form of powder, even more preferably in the form of 95% pure powder. Preferably the plant sterols present and the percentage amount thereof in the phytosterol powder are: betasitosterol (>40%), campesterol (>20%), stigmasterol (>15%), brassicasterol (20%).

Preferably, in the composition as described above, the phytosterols are titrated at least at 95% w/w, preferably at least 98%, with respect to the total of the phytosterol powder, of which preferably at least 80% consists of Beta-sitosterol and beta-sitostanol, 6-10% campesterol, 0.6% stigmasterol, 0.7% delta-7-stigmasterol, 2% campestanol.

The composition as described above can further comprise at least one substance selected from the group consisting of: omega 3, vitamin D, calcium, folic acid, iron, astaxanthin, berberine, coenzyme Q and magnesium.

The composition according to the invention can comprise two or more of the above-described components and all the possible combinations thereof.

In the composition as described above, the components thereof are preferably dehydrated or dry or dried or in powder form.

A further object of the invention is a process for obtaining the composition as described above comprising adding to the Opuntia ficus indica extract at least one substance selected from the group consisting of:
Bergamot (Citrus Bergamia) and/or Lemon and/or Orange and/or Pomelo and/or Grapefruit extract, preferably Bergamot extract,
phytosterols,
and at least one trace element.

In the context of the present invention, the term comprising also includes the term consisting of or characterized by or consisting essentially of. In particular embodiments, such terms are interchangeable.

Said composition is preferably characterized in that said extracts or the composition itself is dehydrated or lyophilized or dry or dried or in the form of powder.

Still more preferably, said composition is characterized in that said extracts are dehydrated in the form of micronized, lyophilized or granulated particles.

The pharmaceutical composition according to the invention or the composition or food supplement according to the invention can be administered in the form of tablets, capsules, oral preparations, oral solutions, powders, granules, pills, injectables, or infusion liquid solutions, suspensions, suppositories, preparation for inhalation. Preferably, the total concentration of the extracts is comprised between 10-90% by weight with respect to the total composition. They are normally presented in unit dose form and contain conventional excipients such as binders, fillers (comprising cellulose, mannitol, lactose), diluents, tablet agents, lubricants (including magnesium stearate), detergents, disintegrants (for example polyvinylpyrrolidone and starch derivatives such as sodium starch glycolate), colouring agents, flavouring agents and wetting agents (for example sodium lauryl sulphate).

The liquid preparations can be for example in aqueous form in oily suspension, solutions, emulsions, syrups or can be presented as a dry product for reconstitution with water. The liquid preparations can contain conventional additives, such as suspension agents, emulsifying agents, non-aqueous vehicles.

A reference for the formulations is the book by Remington (“Remington: The Science and Practice of Pharmacy”, Lippincott Williams & Wilkins, 2000).

The composition, pharmaceutical composition or food supplement according to the invention can be administered in a single dosage containing all the components or as separate (simultaneous or sequential) compositions of the individual components. The composition, pharmaceutical composition or food supplement can be administered in combination with active ingredients which can be formulated separately in single-ingredient preparations of one of the forms described above and then administered as combined preparations which are given at the same time or at different times, or can be formulated together in the same preparation.

The person skilled in the art will choose the form of administration and the effective dosages, by selecting suitable diluents, adjuvants and/or excipients.

Preferably, said composition, pharmaceutical composition or food supplement is in solid form, for example a tablet, a hard capsule, a soft gel capsule, a powder, a syrup, a sachet, a lozenge, a tablet or a pill, small bag or stick pack, or in liquid form, for example an oral spray.

Preferably, said excipient or diluent or pharmaceutically acceptable vehicle is selected from the group consisting of: calcium phosphate, dicalcium phosphate, microcrystalline cellulose, magnesium stearate, silicon dioxide, sucrose, arabic gum, corn starch, medium chain triglycerides, tricalcium phosphate, cross-linked sodium carboxymethylcellulose, hydroxypropylmethylcellulose, polyethylene glycol, titanium dioxide, polyvinylpyrrolidone, talc, erythritol, xylitol, steviol glycosides and sucralose.

The pharmaceutical composition can be chosen based on the treatment to be performed.

Preferably, said pharmaceutical composition is administered orally.

Preferably, said pharmaceutical composition is administered once or twice per day.

The object composition of the present invention, preferably in the form of dry extracts, can be used in the nutraceutical field for example in the form of tablets by addition of at least one excipient, or it can be encapsulated in vegetable gelatin capsules with the addition of at least one pharmaceutically acceptable excipient.

The present composition can be mixed or combined with other active ingredients and/or substances of plant or natural or synthetic origin, and/or vitamins and/or minerals and/or with the addition of excipients or combinations thereof.

Examples of natural substances or ingredients suitable for food or pharmaceutical use preferably present in the present composition are: monacolin k, berberine, artichoke, garlic, polycosanols, Olea europea, plant sterols or stanols, silymarin, green tea extracts, monounsaturated or polyunsaturated fatty acids or chitosan or combinations thereof.

Examples of vitamins preferably present in the present composition are: A, D, E, K, B1, B2, C, Niacin, Pantothenic acid, B6, Folic acid, B12 or Biotin or combinations thereof. These can be in the various possible vitamin formulas.

Examples of minerals preferably present in the present composition are: Calcium, Magnesium, Iron, Copper, Zinc, Iodine, Manganese, Potassium, Sodium, Selenium, Chromium, Molybdenum, Fluoride, Chloride or Phosphorus or combinations thereof. These can be in the form of the various possible mineral substances.

A further object of the present invention is a food supplement or a food product or a drinking product comprising the composition as described above and at least one excipient or diluent and, optionally, a further agent.

Preferably, said food supplement or product or drinking product is for use in the prevention, control and/or treatment of cardio-metabolic disease, dyslipidemia, hypercholesterolemia, hypertriglyceridemia, diabetes associated with dyslipidemia, metabolic syndrome, statin-induced myalgia or myopathy, intolerance to lipid-lowering drugs, clinical conditions characterized by low levels of HDL cholesterol, atherosclerosis, insulin resistance, type II diabetes, NAFLD/NASH, hypertension, obesity, visceral fat, to counteract disorders/risk factors typical of obesity and cardiovascular diseases, such as glycemia, triglycerides, cholesterol.

Preferably, said excipient and/or diluent is selected from the group consisting of: calcium phosphate, dicalcium phosphate, microcrystalline cellulose, magnesium stearate, silicon dioxide, sucrose, arabic gum, corn starch, medium chain triglycerides, tricalcium phosphate, cross-linked sodium carboxymethylcellulose, hydroxypropylmethylcellulose, polyethylene glycol, titanium dioxide, polyvinylpyrrolidone, talc, erythritol, xylitol, steviol glycosides and sucralose.

Preferably, said food supplement or product or drinking product is administered orally. Preferably, said food supplement or product or drinking product is administered once or twice per day.

Preferably, said food supplement or product or drinking product is in the form of a tablet, a hard capsule, a soft gel capsule, a powder, a syrup, a sachet, a lozenge, a tablet, a pill, a food supplement, an edible bar, or an edible snack. In particular, said supplement can be any type of food supplement.

The invention will be illustrated by non-limiting examples.

### EXAMPLES

### Example 1: Preparation Example of an Opuntia ficus indica Extract to be Integrated in the Composition According to the Invention

The cladodes cut from young plants in good vegetative condition are harvested and processed within 24 hours of pruning. After a washing step, they are subjected to a milling process and then extracted with water (1:7 cladodes-water ratio), at a temperature comprised between 15 and 18° C. for 16 hours. A filtration, centrifugation and vacuum concentration step is then carried out. The obtained product is then subjected to precipitation (aqueous extract/isopropanol ratio 1:3), centrifugation, washing, vacuum drying at a temperature of 70° C. Finally, a green-yellow powder is obtained, of variable size around 75-100 microns, composed of at least 20% weight/weight of fiber, of which at least 18% of soluble fibers, and/or composed of at least 75% weight/weight of soluble fiber (mucilage and pectin), containing about 3.7% beta-polysaccharides and 3.7% polyphenols.

Every extract deriving from cladodes of Opuntia ficus indica may be used and allows to obtain similar effects.

### Example 2: Preparation Example of Citrus Bergamia Extract to be Integrated in the Composition According to the Invention

The botanical name of the bergamot extract used is Citrus Bergamia Risso et Poiteau. Such an extract is obtained from the juice of the fruit. The juice of Citrus Bergamia Risso et Poit. is obtained by mechanical pressing of fresh fruits, very ripe, free of rot and mould.

The raw materials used do not contain or derive from genetically modified organisms.

In a next step, the depulpation, pasteurization and collection in aseptic containers are carried out.

The juice thus obtained undergoes a filtration, extraction and concentration process.

In particular, the juice is passed through specific membranes and subjected to successive microfiltration and nanofiltration steps, which allow the passage of water, ions and water-soluble compounds (such as flavonoids) while excluding the passage of microorganisms such as viruses and bacteria.

The next process consists in the passage of the filtered juice through specific non-polymeric adsorbent resins (to avoid the release of low molecular weight polymers, potentially harmful to health) which allow to obtain an Bergamia Risso et Poit. citrus juice extract which is highly rich in flavonoids.

The juice obtained is finally subjected to a spray-drying process which allows the elimination of water and obtaining a dry extract. The extract is then titrated using HPLC (high performance liquid chromatography).

### Example 4: Preparation Example of Phytosterols

The phytosterols used in the composition object of the invention are obtained by processing pine logs (Pinus spp) using n-hexane, ethanol and methanol as extraction solvents. The appropriately crushed pine log-derived starting material is mixed in a 1:2 ratio with the solvent mixture at room temperature. The solvent is then removed through distillation. The preparation obtained is presented as a fine white powder, the titre of which (expressed in % weight/weight) in total phytosterols determined with HPLC is at least 98%, of which at least 80% consists of Beta-sitosterol and beta-sitostanol, 6-10% campesterol, 0.6% stigmasterol, 0.7% delta-7-stigmasterol, 2% campestanol.

### Example 5: Example Composition According to the Invention in the Form of Swallowable Tablets

In the present example, the composition object of the present invention is in the form of swallowable tablets. It comprises the following ingredients, as a percentage of the total weight of the composition: opuntia extract 12.5%, bergamot extract 16.6%, pine phytosterols 33.3%, thiamine 100 in addition to excipients such as calcium phosphate, magnesium stearate, silicon dioxide, hydroxypropylcellulose, mono and diglycerides of fatty acids, microcrystalline cellulose, coating agents, iron oxide dye.

| | | Nutritional values of |
|---|---|---|
| List of ingredients | mg per 1 tablet | reference* per 1 tablet |
| Phytosterols | 400 | — |
| Opuntia extract | 150 | — |
| Bergamot extract | 200 | — |
| Thiamine (thiamine | 12.5 | 1136.3% |
| hydrochloride) | | |

*Annex XIII Reg (EU) 1169/2011

### Example 6: Example Composition According to the Invention in the Form of a Powder in Sachet

In the present example, the composition object of the present invention is in the form of a powder in sachet to be dissolved in water.

It contains the following ingredients, as a percentage of the total weight of the composition: opuntia extract 20, phytosterols 40%, citrus extract mixture (bergamot, grapefruit, pomelo) 10%, thiamine 0.50 as well as excipients such as maltodextrins, silicon dioxide, steviol glycosides as a sweetener.

| | | Nutritional values of |
|---|---|---|
| List of ingredients | mg per 1 sachet | reference* per 1 sachet |
| Phytosterols | 2000 | — |
| Opuntia extract | 1000 | — |
| Citrus extract mixture | 500 | — |
| Thiamine (thiamine | 25 | 2272.7% |
| hydrochloride) | | |

*Annex XIII Reg (EU) 1169/2011

### Example 7: Example Composition According to the Invention in Liquid Form in Vial

In the present example, the composition object of the invention is presented in the form of a beverage in a vial, containing the following ingredients, as a percentage of the total weight of the composition, per single dose: opuntia extract 6%, phytosterols 16%, bergamot extract 8%, thiamine 0.5% in addition to excipients such as water, thickeners, sweeteners, dyes, citric acid as an acidifier, sodium benzoate and potassium sorbate as preservatives.

| | | Nutritional values of |
|---|---|---|
| List of ingredients | mg per 1 vial | reference* per 1 vial |
| Phytosterols | 800 | — |
| Opuntia extract | 300 | — |
| Bergamot blend | 400 | — |
| Thiamine (thiamine | 25 | 2272.7% |
| hydrochloride) | | |

*Annex XIII Reg (EU) 1169/2011

### Example 8: Example Composition According to the Invention in the Form of Swallowable Tablets

In the present example, the composition object of the present invention is in the form of swallowable tablets. It comprises the following ingredients, as a percentage of the total weight of the composition: opuntia cladodes extract 50%, bergamot extract 20%, in addition to excipients such as calcium phosphate, magnesium stearate, silicon dioxide, hydroxypropylcellulose, mono and diglycerides of fatty acids, microcrystalline cellulose, coating agents, iron oxide dye

| | | Nutritional values of |
|---|---|---|
| List of ingredients | mg per 1 tablet | reference* per 1 tablet |
| Opuntia extract | 500 | — |
| Bergamot extract | 200 | — |

### Example 9: Example Composition According to the Invention in the Form of a Powder in Sachet

In the present example, the composition object of the present invention is in the form of a powder in sachet to be dissolved in water.

It contains the following ingredients, as a percentage of the total weight of the composition: opuntia cladodes extract 12.5%, phytosterols 50%, as well as excipients such as maltodextrins, silicon dioxide, steviol glycosides as a sweetener.

| | | Nutritional values of |
|---|---|---|
| List of ingredients | mg per 1 sachet | reference* per 1 sachet |
| Opuntia extract | 500 | — |
| Phytosterols | 2000 | — |

### Example 10: Clinical Evaluation of the Efficacy of the Composition

A single-centre, 6-arm, double-blind, placebo-controlled, pilot clinical study was conducted to evaluate the efficacy of the composition object of the present invention on humans in reducing hyperlipidemia-related risk factors. The study was carried out in accordance with the guidelines of the Helsinki Declaration and approved by the Ethics Committee of the Hospital. 90 adult individuals of both sexes were enrolled (47 women and 43 men, aged 20 to 75 years) with moderate hypercholesterolaemia (LDL-cholesterol between 115 and 190 mg/dL) in primary prevention for cardiovascular diseases.

Exclusion criteria: hypercholesterolaemia secondary to pathological or iatrogenic causes, hypertriglyceridaemia (Triglycerides>400 mg/dL), type I or II diabetes, systemic diseases associated with dyslipidemia, uncompensated dysthyroidism, treatment with drugs potentially interacting with lipid metabolism (corticosteroids, atypical antipsychotics).

The subjects enrolled were randomized into 6 groups, each consisting of 15 individuals:
Group A: 1 tablet of active ingredient (COMP)
Group B: 1 tablet of placebo
Group C: 1 tablet of bergamot extract 200 mg
Group D: 1 tablet of opuntia extract 150 mg
Group E: 1 table of phytosterols 400 mg
Group F: 1 tablet of thiamine (thiamine hydrochloride) 12.5 mg

All the enrolled subjects were subjected, 14 days prior to enrolment (T-14), to a pre-enrolment visit with blood tests to assess meeting the inclusion criteria. The individuals who at TO met the inclusion criteria underwent a randomization visit at which the respective treatment was assigned and blood tests were performed in relation to the reference parameters.

The study lasted 12 weeks.

At the end of treatment (T12), the subjects were subjected to an end-of-study visit and blood tests. The parameters investigated in the study are: Body mass index, systolic and diastolic blood pressure, blood glucose, total cholesterol, LDL, HDL, triglycerides, GOT, GPT, gGT, creatinine phosphokinase (CPK).

The treatment called “active” or “COMPOSITION (COMP)” consists of a 1200 mg tablet consisting of 150 mg of Opuntia ficus indica extract, 200 mg of bergamot extract, 400 mg of phytosterols, 12.5 mg of vitamin B1 (thiamine as thiamine hydrochloride) in addition to excipients such as calcium phosphate, magnesium stearate, silicon dioxide, hydroxypropyl cellulose, mono- and di-glycerides of fatty acids, microcrystalline cellulose, yellow iron oxide as a dye, yellow coating agents. The treatment called “placebo” consists of a tablet equal in shape, weight, colour size and flavour to the active tablet but containing “inert” material for the desired effect and consists of: microcrystalline cellulose, magnesium stearate, silicon dioxide, calcium phosphate, chlorophylline dye, yellow iron oxide dye, yellow coating agents.

The treatments related to groups C, D, E and F consist of one tablet equal in appearance to the tablets of groups A and B but consisting of the single active ingredient, as described above in addition to excipients.

The treatment tablets were packaged in anonymous white bottles, showing only the batch number consisting of an alphanumeric code and the expiration date.

Neither the investigator nor the study subjects knew the contents of the bottles. The bottles of each treatment were placed in cardboard boxes containing a letter in a sealed envelope with reference to the batch number and content, to be opened only in case of need, as in cases of serious adverse events. After randomization, each study participant was given the vial with 30 tablets for the specific treatment of the group to which they belonged.

All the subjects were instructed to take one tablet in the evening before bed for 12 weeks.

The results are described in table 1.

After 12 weeks of treatment, groups A and C achieved statistically significant reductions both respect to the baseline and to the placebo group of the following parameters: total cholesterol (TC), LDL-cholesterol (LDL-c), triglycerides (TG), fasting plasma glucose (FPG) and a statistically significant increase in both baseline and placebo HDL cholesterol.

The groups B, E, F did not show statistically significant reductions in the parameters of interest.

Group D showed a statistically significant reduction in fasting plasma glucose (FPG).

There were no significant changes in systolic and diastolic blood pressure, body mass index, creatine phosphokinase (CPK), GOT transaminases, GPT, gammaGT, for any of the treatment groups.

In particular, the results of group A were significantly higher with respect to the individual groups C and E and higher with respect to the sum of the individual results of groups C, D, E, F, showing a synergistic effect of the composition object of the present invention.

Regarding the results related to total cholesterol (TC), group A achieved a 25% reduction while group C 11%, group D 2%, group E 5% and F null. The sum of the reductions of the individual actives is equal to 18%, thus lower than the result obtained with the composition object of the invention.

Regarding LDL cholesterol (LDL-C), group A achieved a reduction of 34%, group C of 21%, group D of 3% and group E of 5% (sum −29%). Group F did not achieve a percentage reduction in LDL cholesterol. The sum of the reductions of the individual actives is equal to 29%, thus lower than the result obtained with the composition object of the invention.

In the variations of HDL cholesterol (HDL-C), group A showed an increase of 18%, group C of 9%, group D of 2%, group E a reduction of 2% and group F showed no changes, thus the sum of the percentage increases of the individual compounds is equal to 9%, lower with respect to the group treated with the composition object of the invention.

In the changes in triglycerides (TG), group A showed a statistically significant reduction of 30%, therefore higher than the sum of the reductions of the treatment groups with the individual substances, equal to −26% (−17% for group C, −4% for group D, −4% for group E, −1% for group F).

Finally, also with regard to changes in fasting plasma glucose (FPG), group A achieved a reduction of 36%, higher than the sum of the reductions of groups C (−11%), D (−16%), E and F (the latter not detected).

Compliance was optimal in all the treatment groups. No adverse events or increase in transaminases or CPK were detected in either group A or the other treatment groups, emphasizing the safety of the inventive composition.

TABLE 1
Changes in parameters investigated during the study.

| | GROUP A | | Group B | | Group | | Group D | | Group E | | Group F | |
|---|---|---|---|---|---|---|---|---|---|---|---|---|
| | (COMP) | | (PLACEBO) | | (BERGAMOT) | | (OPUNTIA) | | (PHYTOSTEROLS) | | (THIAMINE) | |
| | (n = 15; | | (n = 15; | | (n = 15; | | (n = 15; | | (n = 15; | | (n = 15; | |
| | F = 7, M = 8) | | F = 7, M = 8) | | F = 9, M = 6) | | F = 8, M = 6) | | F = 9, M = 7) | | F = 7, M = 8) | |
| | | 12- | | 12- | | 12- | | 12- | | 12- | | 12- |
| | | Week | | Week | | Week | | Week | | Week | | Week |
| | | follow- | | follow- | | follow- | | follow- | | follow- | | follow- |
| | Baseline | up | Baseline | up | Baseline | up | Baseline | up | Baseline | up | Baseline | up |
| BMI | 24.0 ± | 23.7 ± | 23.8 ± | 23.4 ± | 24.5 ± | 24.0 ± | 24.0 ± | 23.9 ± | 23.8 ± | 23.7 ± | 24.0 ± | 23.9 ± |
| (kg/m²) | 1.8 | 1.7 | 1.7 | 1.4 | 1.9 | 1.1 | 1.8 | 1.6 | 1.7 | 1.5 | 1.6 | 1.3 |
| SBP | 131 ± | 130 ± | 128 ± | 126 ± | 126 ± | 124 ± | 130 ± | 129 ± | 131 ± | 130 ± | 129 ± | 127 ± |
| (mmHg) | 7 | 7 | 8 | 6 | 9 | 7 | 7 | 6 | 5 | 7 | 7 | 7 |
| DBP | 84 ± | 83 ± | 85 ± | 84 ± | 84 ± | 83 ± | 84± | 83 ± | 85 ± | 84 ± | 84 ± | 83 ± |
| (mmHg) | 5 | 6 | 6 | 5 | 7 | 6 | 6 | 7 | 6 | 4 | 5 | 6 |
| TC | 253 ± | 189 ± | 248 ± | 249 ± | 255 ± | 226 ± | 250 ± | 245 ± | 252 ± | 239 ± | 250 ± | 250 ± |
| (mg/dL) | 15 | 9*° | 12 | 10 | 13 | 9*° | 13 | 10 | 11 | 15 | 13 | 9 |
| LDL-C | 176 ± | 116 ± | 171 ± | 174 ± | 167 ± | 131 ± | 177 ± | 171 ± | 172 ± | 163 ± | 167 ± | 167 ± |
| (mg/dL | 10 | 6*° | 9 | 11 | 12 | 7*° | 9 | 12 | 10 | 11 | 12 | 8 |
| HDL-C | 44 ± | 52 ± | 45 ± | 45 ± | 46 ± | 50 ± | 44 ± | 45 ± | 46 ± | 45 ± | 43 ± | 43 ± |
| (mg/dL | 4 | 3*° | 2 | 3 | 3 | 4*° | 3 | 2 | 3 | 3 | 3 | 1 |
| TG | 165 ± | 115 ± | 159 ± | 160 ± | 151 ± | 124 ± | 151 ± | 145 ± | 159 ± | 152 ± | 161 ± | 159 ± |
| (mg/dL) | 11 | 12*° | 12 | 10 | 13 | 12*° | 13 | 9 | 12 | 10 | 11 | 13 |
| FPG | 118 ± | 75 ± | 110 ± | 108 ± | 110 ± | 98 ± | 115 ± | 96 ± | 111 ± | 112 ± | 118 ± | 118 ± |
| (mg/dL) | 9 | 6*° | 6 | 9 | 8 | 5*° | 6 | 8*° | 5 | 4 | 6 | 4 |
| GOT | 21 ± | 21 ± | 18 ± | 17 ± | 21 ± | 20 ± | 21 ± | 20 ± | 19 ± | 18 ± | 20 ± | 19 ± |
| (U/L) | 6 | 5 | 6 | 5 | 7 | 5 | 6 | 5 | 5 | 7 | 5 | 4 |
| GPT | 23 ± | 22 ± | 21 ± | 20 ± | 22 ± | 22 ± | 21 ± | 22 ± | 23 ± | 22 ± | 22 ± | 21 ± |
| (U/L) | 7 | 7 | 5 | 6 | 6 | 6 | 5 | 4 | 6 | 7 | 6 | 6 |
| Gamma-GT | 28 ± | 25 ± | 25 ± | 24 ± | 26 ± | 23 ± | 25 ± | 24 ± | 24 ± | 27 ± | 25 ± | 23 ± |
| (U/L) | 9 | 9 | 7 | 6 | 9 | 6 | 6 | 5 | 9 | 6 | 6 | 4 |
| CPK | 121 ± | 127 ± | 134 ± | 130 ± | 129 ± | 131 ± | 132 ± | 131 ± | 134 ± | 133 ± | 129 ± | 131 ± |
| (U/L) | 23 | 38 | 42 | 28 | 37 | 21 | 27 | 27 | 25 | 22 | 41 | 27 |

*P < 0.05 vs. baseline;
°P < 0.05 vs placebo.

In order to assess the efficacy of the composition of the present invention (herein defined as “COMP”) and in particular the key role of the Opuntia on top of a composition made of bergamot and phytosterols, a double-blind study was performed in 40 healthy people with moderate hypercholesterolemia.

Subjects (24 males, 16 women; age between 18 and 69 years) were randomized and assigned to the relevant treatment.

GROUP A: 20 people took 1 capsule per day, composed of 200 mg of bergamot juice dry extract, 400 of Phytosterols and 150 mg of Opuntia cladodes extract for 12 weeks.

GROUP B: 20 people took 1 capsule per day, composed of 200 mg of bergamot juice dry extract, 400 mg of Phytosterols for 12 weeks.

Capsules were identical in size and color and non-distinguishable.

Parameters monitored were: total cholesterol (t-chol) LDL-cholesterol (LDL-c), HDL-cholesterol (HDL-c), triglycerides (TG), Fasting plasma glucose (FPG).

Results are described in Table 2.

TABLE 2
Changes in parameters investigated during the study (2).

| | GROUP A | | | GROUP B | | |
|---|---|---|---|---|---|---|
| | (COMP) | | | (bergamot + phytosterols) | | |
| | (n = 20) | | | (n = 20) | | |
| | | 12-Week | % change vs | | 12-Week | % change vs |
| | Baseline | follow-up | baseline | Baseline | follow-up | baseline |
| TC | 270 ± 9 | 203 ± 7* | −25% | 268 ± 7 | 227 ± 8* | −15% |
| (mg/dL) | | | | | | |
| LDL-C | 181 ± 11 | 119 ± 8* | −34% | 174 ± 9 | 142 ± 8* | −18% |
| (mg/dL | | | | | | |
| HDL-C | 48 ± 6 | 57 ± 5* | +18% | 45 ± 2 | 47.7 ± 3* | +6% |
| (mg/dL | | | | | | |
| TG | 169 ± 9 | 118 ± 9* | −30% | 158 ± 12 | 123 ± 10* | −22% |
| (mg/dL) | | | | | | |
| FPG | 122 ± 9 | 78 ± 6* | −36% | 110 ± 6 | 105 ± 9* | −5% |
| (mg/dL) | | | | | | |

*P < 0.05 vs. baseline.

These results show an unexpected increased efficacy of the Composition of opuntia plus bergamot and phytosterols versus bergamot and phytosterols only.

Moreover, the effect obtained on the investigated parameters show a % of change that is higher than the sum of GROUP B of this study plus GROUP D of Study 1 of this paper (treatment with Opuntia extract only) thus showing a synergistic effect of the combination of Bergamot, Opuntia and Phytosterols.

A similar experiment was performed also with a group of subjects taking a product composed of Bergamot 200 mg+Opuntia extract 500 mg daily for 12 weeks. Results in terms of reduction of total cholesterol (−25%), LDL cholesterol (−32%), TG (−35%) and FPG (−37%) and an increase of HDL-c of 16% vs baseline, were similar to what obtained with the composition of group A of the previous example.

1. A composition comprising Opuntia ficus indica extract and a substance selected from the group consisting of:
Bergamot (Citrus Bergamia) and/or Lemon and/or Orange and/or Pomelo and/or Grapefruit extract,
phytosterols,
and a trace element.

2. The composition according to claim 1 comprising Opuntia ficus indica extract and Citrus Bergamia extract or Opuntia ficus indica extract and phytosterols.

3. The composition according to claim 1 comprising Opuntia ficus indica extract, Citrus Bergamia extract and phytosterols.

4. The composition according to claim 1 comprising Opuntia ficus indica extract, Citrus Bergamia extract, phytosterols and trace elements.

5. The composition according to claim 1 wherein the Citrus Bergamia extract comprises brutieridin and melitidin.

6. The composition according to claim 1, wherein the Opuntia Ficus Indica extract derives from cladodes, flowers, fruits or combinations thereof.

7. The composition according to claim 1 wherein the Opuntia ficus indica extract comprises:
at least 20% weight/weight of fiber, of which at least 18% of soluble fiber, or
at least 70% of soluble fiber, and optionally wherein said soluble fiber comprises 3-5% of beta-polysaccharides and 3-5% of polyphenols.

8. The composition according to claim 1 wherein the phytosterols comprise about 80% of beta-sitosterol and beta-sitostanol, 6-10% of campesterol, 0.5-1% of stigmasterol, 0.5-1% of delta-7-stigmasterol, 2% of campestanol.

9. The composition according to claim 1 wherein the trace element comprises a vitamin and/or a mineral, and the trace element is optionally thiamine and/or its salts.

10. The composition according to claim 1 comprising:
a. Opuntia ficus indica extract from 5 to 20% w/w
b. Citrus Bergamia extract from 8 to 20% w/w
c. Phytosterols from 10 to 40% w/w
d. Thiamine hydrochloride from 0.5 to 1% w/w.

11. The composition according to claim 1 wherein the composition is in solid, semi-solid or liquid form and/or is optionally in combination with one or more excipients selected from the group comprising: calcium phosphate, magnesium stearate, silicon dioxide, hydroxypropylcellulose, mono and diglycerides of fatty acids, microcrystalline cellulose, glazing agents, maltodextrins, acidifiers, preservatives, sweeteners, dyes; and/or in combination with other ingredients suitable for food or pharmaceutical use.

12. The composition according to claim 1 wherein:
a. Opuntia ficus indica extract is present in an amount from 150 mg to 1000 mg;
b. Citrus Bergamia extract is present in an amount from 200 mg to 500 mg;
c. Phytosterols are present in an amount from 400 to 2000 mg;
d. Thiamine hydrochloride is present in an amount from 10 to 25 mg.

13. The pharmaceutical composition comprising the composition according to claim 1 and at least one pharmaceutically acceptable excipient and/or vehicle.

14. A pharmaceutical composition, dietary supplement or food product or drinking product comprising the composition according to claim 1.

15. A method for the reduction and prevention of risk factors related to the development of cardio-metabolic diseases comprising administering a composition of claim 1 to a patient in need thereof.

16. (canceled)

17. The composition of claim 12, wherein the substance is Bergamot extract.

18. The composition of claim 12, wherein the Opuntia ficus indica extract is present in an amount of 150 mg, 300 mg or 1000 mg.

19. The composition of claim 12, wherein the Citrus Bergamia extract is present in an amount of 200 mg 400 mg or 500 mg.

20. The composition of claim 12, wherein the Phytosterols are present in an amount of 400 mg, 800 mg or 2000 mg.

21. The composition of claim 12, wherein the Thiamine hydrochloride is present in an amount of 12.5 mg or 25 mg.
